(19) United States
(12) Patent Application Publication
(10) Pub. No.: US 2019/0173256 A1
(43) Pub. Date: Jun. 6, 2019

(54) FEMTOSECOND LASER SOURCE

(71) Applicants: CENTRE NATIONAL DE LA RECHERCHE SCIENTIFIQUE -CNRS-, PARIS (FR); UNIVERSITE DE LIMOGES, LIMOGES (FR)

(72) Inventors: Sébastien FEVRIER, Bosmie-L'Aiguille (FR); Leonid KOTOV, LIMOGES (FR); Ammar HIDEUR, Le Petit Quevilly (FR)

(21) Appl. No.: 16/320,993

(22) PCT Filed: Jul. 25, 2017
(86) PCT No.: PCT/EP2017/068710

§ 371 Date: Jan. 25, 2019

(30) Foreign Application Priority Data
Jul. 26, 2016 (FR) 1657179

(51) Int. Cl. H01S 3/067; A61B 5/00; G01N 21/64; H01S 3/16; H01S 3/30

(52) U.S. Cl. CPC H01S 3/06725; H01S 3/06754; A61B 5/0071; H01S 2301/085; H01S 3/1608; H01S 3/302; G01N 21/6458

(57) ABSTRACT

A femtosecond laser source includes an injection laser oscillator with an optical fiber doped with a given material, suitable for delivering, via an output optical fiber, a first picosecond pulse, at a first wavelength λ₁; a power amplifier with an amplifying optical fiber for producing, from the first pulse, a second pulse at the first wavelength, with an energy that is amplified relative to the first pulse, the amplifying optical fiber being doped with the same material as the optical fiber of the injection oscillator and having a length less than or equal to the distance from the point of soliton compression and greater than the distance from which the amplifying optical fiber operates in non-linear mode; a fiber with a frequency shift suitable for receiving the second pulse and generating, by Raman self-shifting, a fundamental soliton at a second wavelength λ₂ that is strictly greater than the first wavelength λ₁.

### TECHNICAL FIELD

The present description relates to a femtosecond laser source and to a method for generating femtosecond laser pulses. It more particularly relates to a femtosecond laser source suitable for multi-photon microscopy for imaging biological tissues.

### PRIOR ART

Multi-photon microscopy allows images of biological tissues to be produced in vivo or ex vivo and has applications for example in the fields of the neurosciences, of embryology or of oncology. Thus, commercial multi-photon microscopes use the interaction of two photons with the molecules of an organic medium to locate organelles therein and to trace, by collecting the emitted fluorescence, a dynamic map of these organelles. The best commercial microscopes allow tissues to be visualized at 200 μm under the surface of the tissues. Using the interaction of three photons with tissues, it is possible to visualize tissues located at greater depth (>800 μm) with the same signal-to-noise ratio.

FIG. 1 illustrates various characteristic lengths of the response of disorganized aqueous tissues, such as those of the cerebral cortex, in the presence of laser pulses. These characteristic lengths depend on the wavelength of the pulses. In FIG. 1, curve 12 represents the characteristic absorption length (lₐ) in water, which is defined as the length required for the optical power of the incident signal to be attenuated by a factor e, where e is Euler's number; the curve 13 represents the characteristic scattering length (l_{d}) and the curve 11 represents an effective attenuation length (l_{eff}) that takes into account the absorption and scattering (l_{eff}⁻¹=lₐ⁻¹+l_{d}⁻¹). These various curves, and in particular curve 11, illustrate the fact that, in a window 14 of wavelengths about 1675 nm (for example, 1675 nm +/−25 nm), laser pulses penetrate more deeply into aqueous tissues.

The applicant has more precisely observed that, for imaging at depth in biological media, a laser source allowing laser pulses of high peak power (typically of about 100 kW or more) and of duration shorter than 200 femtoseconds is ideally required, on account of the properties of disorganized aqueous tissues such as biological media, in addition to an optimized wavelength close to 1675 nm. Such a laser source coupled to a microscope allows images to be acquired at depth in a biological medium with a good signal-to-noise ratio.

Laser sources allowing ultra-brief pulses having such a peak power do exist; these laser sources consist of a succession of elements, for example comprising an oscillator-injector, a stretcher, one or more amplifiers, a compressor and a wavelength-converting element.

The document entitled “In vivo three-photon microscopy of subcortical structures within an intact mouse brain”, by Horton et al., published Jan. 20, 2013 in Nature Photonics, thus describes a microscopy technique, applicable to in vivo imaging, in particular for producing in vivo images of mouse brain. High-energy pulses are produced at a wavelength of 1675 nm by means of a photonic crystal rod by self-frequency shifting from input laser-source pulses of 1550 nm wavelength. On exiting, each pulse has a peak power of 1 megawatt (MW) and a full width at half intensity maximum of 114 femtoseconds. The pulse train has a repetition rate of 1 megahertz (MHz), this corresponding to an average power of 67 milliwatt (mW).

However, in known prior-art sources, when the laser beam passes from one element to the next, by way of free-space paths, it is necessary to collimate then focus this laser beam in each of the amplifying elements and/or wavelength-converting elements, whether they consist of non-linear crystals or fibers. Thus, maintenance and adjustment of such laser sources must be carried out regularly, this possibly being constraining for the users of these laser sources, in particular when these users do not necessarily have the time or equipment, or even the knowledge to carry out such maintenance.

There thus appears to be a need for a laser source able to provide a performance suitable for producing images at depth in living tissues and the maintenance of which is simplified.

### SUMMARY

The subject of the present description, according to a first aspect, is a femtosecond laser source comprising:
an injecting laser oscillator based on optical fiber doped with a given dopant, suitable for delivering, via an exit optical fiber, a first picosecond pulse, at a first wavelength λ₁;
a power amplifier based on amplifying optical fiber for producing, from the first pulse, a second pulse, at the first wavelength, with an energy that is amplified with respect to the first pulse, the amplifying optical fiber being doped with the same dopant as the optical fiber of the injecting laser oscillator, and having a length smaller than or equal to the distance of the soliton compression point and larger than the distance from which the amplifying optical fiber operates in non-linear regime; and
a frequency-shifting fiber suitable for receiving the second pulse and generating by Raman self-shifting a fundamental soliton at a second wavelength λ₂ strictly longer than the first wavelength λ₁.

Because of the combined use of an amplifying fiber doped with the same dopant as the fiber of the injecting laser oscillator and of a frequency-shifting fiber, it is possible to obtain a train of pulses that are both ultra-brief (of duration for example shorter than 100 femtoseconds) and of high peak power (higher for example than 100 kW), with a high repetition rate (for example between 0.1 and 100 MHz). In the rest of the description, femtosecond pulses will be spoken of when referring to pulses of duration shorter than 100 femtoseconds.

Furthermore, the second wavelength depending on the dopant and on the length of the frequency-shifting fiber, it is possible to obtain pulses at a second wavelength that is suitable for a given application, for example for the imaging applications mentioned in the introduction.

The use and maintenance of such a laser source are simplified in that the laser source lends itself to production in the form of a monolithic device, in which the various optical components (laser oscillator, power amplifier and shifting fiber) are integrated together, connected via the optical fibers and do not require adjustments. The laser source is furthermore stable over time and immune to environmental perturbations such as mechanical vibrations.

In at least one embodiment of the laser source, the amplifying optical fiber has a modal area larger than or equal to 200 λ₁². Such a modal area increases the peak power of the pulse produced by the amplifying optical fiber.

In at least one embodiment of the laser source, the amplifying optical fiber has a length larger than the distance from which the amplifying optical fiber operates in non-linear regime. Thus, non-linear effects such as self-phase modulation allow the pulses to be temporally compressed and the peak power able to be obtained as output from the amplifying fiber to be further increased.

In at least one embodiment of the laser source, the frequency-shifting fiber has a modal area larger than that of the amplifying fiber. Such a modal combination allows the peak power of the pulse obtained as output from the laser source to be notably increased.

In at least one embodiment of the laser source, the injecting laser oscillator is a fiber oscillator with chromatic dispersion management and that is configured to generate frequency-chirped pulses. Such pulses have a substantial frequency dispersion and may thus be amplified more than unchirped pulses.

In at least one embodiment of the laser source, the injecting laser oscillator comprises a soliton oscillator that delivers pulses that are not frequency chirped.

In at least one embodiment of the laser source, the soliton oscillator is followed by a normal-dispersion optical fiber configured to temporally stretch the pulses generated by the oscillator.

In at least one embodiment of the laser source, the dopant is a rare earth chosen from ytterbium, praseodymium, erbium, thulium and holmium. The dopant may also be bismuth. These materials make it possible to achieve, as output from the frequency-shifting fiber, pulses that have a wavelength suitable for the aforementioned imaging applications.

In at least one embodiment of the laser source, the amplifying optical fiber and the frequency-shifting fiber are spliced to each other. Furthermore, the output optical fiber of the injecting laser oscillator and the amplifying fiber are spliced to each other. The laser source is thus monolithic and does not require adjustment. Air paths are also avoided.

In at least one embodiment, the laser source furthermore comprises a high-pass filter for removing pulse residues at the first wavelength λ₁. This allows the effectiveness of the pulses produced by the laser source in the aforementioned imaging applications to be increased.

The subject of the present description, according to a second aspect, is an imaging system comprising a femtosecond laser source according to the present description, suitable for emitting pulses toward an object located at depth in a biological medium and a microscope for forming and acquiring an image of the object from fluorescence light backscattered by the object.

The subject of the present description, according to a third aspect, is a method for generating femtosecond laser pulses comprising producing, with an injecting oscillator based on optical fiber doped with a given dopant, a first picosecond pulse, at a first wavelength λ₁; producing, with a power amplifier based on amplifying optical fiber, from the first pulse, a second pulse, at the first wavelength, with an energy that is amplified with respect to the first pulse, the amplifying optical fiber being doped with the same dopant as the optical fiber of the injecting oscillator, and having a length smaller than or equal to the distance of the soliton compression point; and producing, by Raman self-shifting, by means of a frequency-shifting fiber suitable for receiving the second pulse, a fundamental soliton at a second wavelength λ₂ strictly longer than the first wavelength λ₁.

### BRIEF DESCRIPTION OF THE FIGURES

Other advantages and features of the technique/technology presented above will become apparent on reading the description that is detailed below, which is given with reference to the figures, in which:

FIG. 1, which has already been described, is a graph illustrating characteristics of the response of aqueous tissues in the presence of a laser source;

FIGS. 2A-2B illustrate embodiments of a laser source according to the present description;

FIGS. 3A-3C illustrate properties of the laser pulses generated by the laser source according to the present description;

FIGS. 4A-4E illustrate various aspects of the soliton-compression effects;

FIGS. 5A-5C illustrate various aspects of the effect of Raman self-shifting on solitons;

FIGS. 6A-6B illustrate properties of a laser source according to the present description;

FIGS. 7A-7B illustrate properties of a laser source according to the present description; and

FIG. 8 is an image obtained with an imaging system according to the present description.

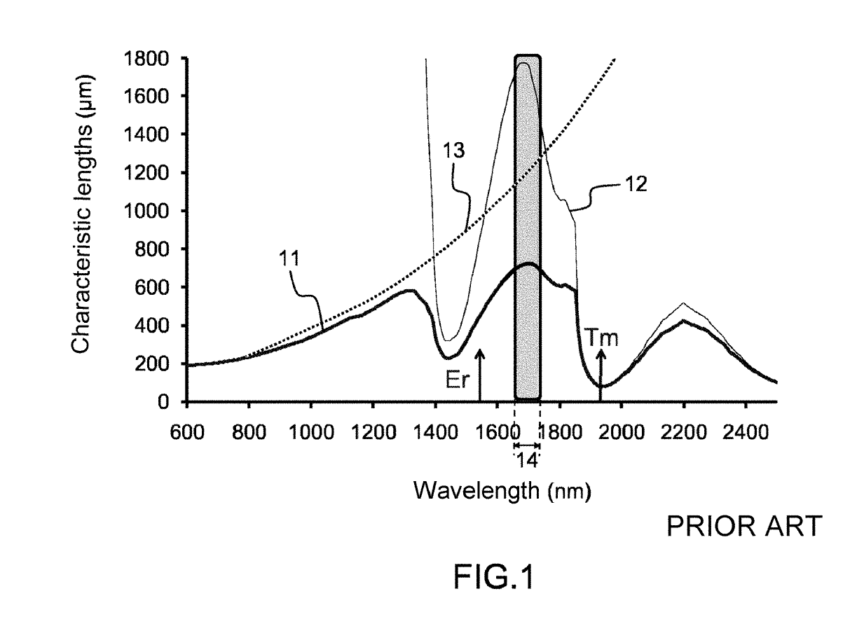

### DETAILED DESCRIPTION

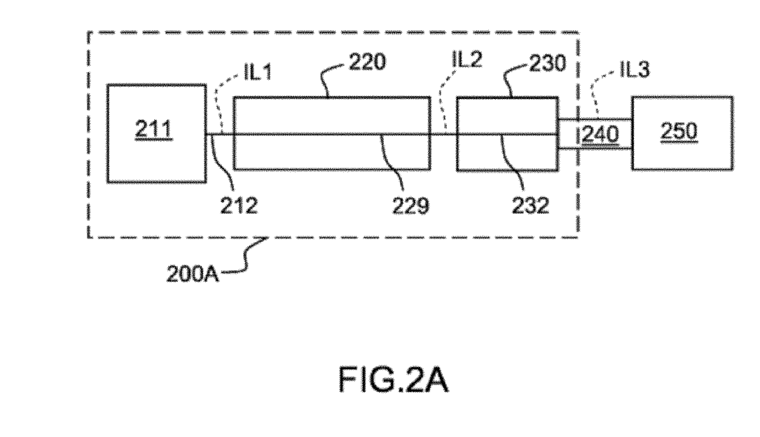

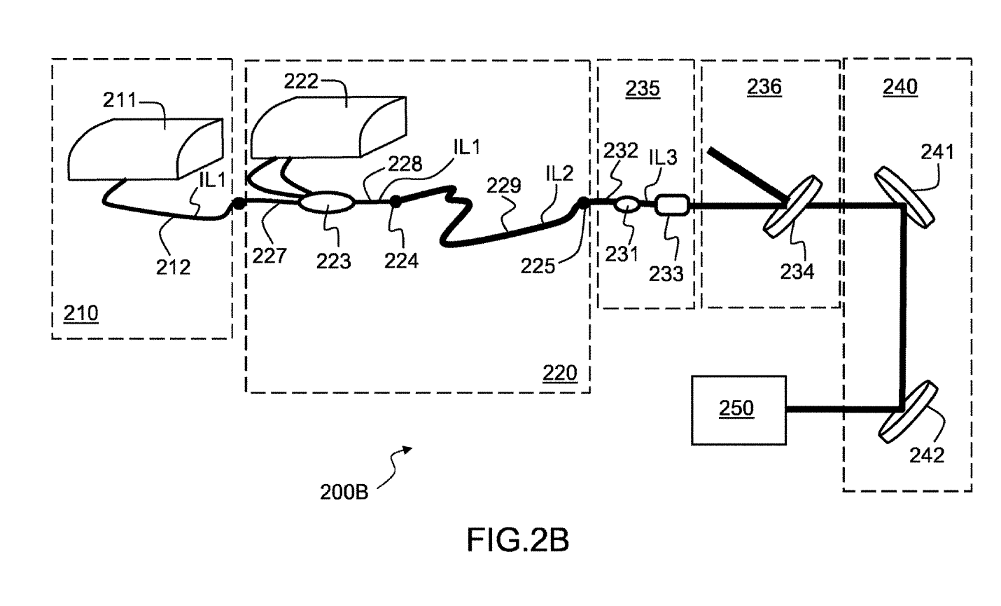

FIG. 2A schematically shows one embodiment of a laser source 200A according to the present description, which embodiment is for example suitable for multiphoton microscopy, and FIG. 2B shows a more detailed example embodiment of a laser source 200B according to the present description. The laser source 200A or 200B is a device for generating laser pulses.

The laser source 200A shown in FIG. 2A comprises three main components, namely:
an injecting laser oscillator 211,
a power amplifier 220 based on optical fiber 229, and
a frequency-shifting device 230 comprising an optical fiber 232.

The laser source 200A generates a train of optical pulses that are conveyed, by means of an exit optical component 240, toward a microscope 250 allowing images to be formed and acquired.

The injecting laser oscillator 211 comprises at least one optical fiber that is doped with a given dopant. The doped optical fiber of the injecting laser oscillator 211 is for example an optical fiber made of a given luminescent material (glass or vitreous matrix) doped with a given dopant. The dopant is an optically active material, i.e. a material that, under excitation (for example by pumping lasers internal to the laser oscillator 211), emits coherent light at a given wavelength. In one or more embodiments, this doped optical fiber is an amplifying fiber internal to the injecting laser oscillator 211. In one or more embodiments, the dopant is an ion, for example a rare-earth ion. The rare earth is for example neodymium (of chemical symbol Nd), ytterbium (of chemical symbol Yb), praseodymium (of chemical symbol Pr), erbium (of chemical symbol Er), thulium (of chemical symbol Tm), holmium (of chemical symbol Ho), or any other fluorescent element that is soluble in the vitreous matrix forming the fiber, such as for example bismuth (of chemical symbol Bi).

The injecting laser oscillator 211 produces as output, via an exit optical fiber 212, a first train of laser pulses IL1, at a first wavelength λ₁. The laser pulses IL1 are picosecond (ps) pulses. In the context of the present description, a picosecond pulse is a pulse of duration comprised between 1 and 100 ps. The repetition rate of the laser pulses IL1 is for example comprised between 0.1 and 100 MHz.

In at least one embodiment of the laser source 200A, the injecting laser oscillator 211 is a phase-wise mode-locked fiber laser oscillator. Such locking of the longitudinal modes of the injecting laser oscillator allows picosecond pulses to be obtained. Other types of lasers, for example gain-switched lasers, also allow picosecond pulses to be obtained.

The first wavelength λ₁ depends on the dopant of the doped optical fiber of the injecting laser oscillator 211. When the dopant is erbium, the first wavelength λ₁ is about 1555 nm. More generally, depending on the chosen dopant, the wavelength λ₁ may be comprised between 900 and 2200 nm.

The power amplifier 220 generates, from the first train of laser pulses IL1, a second train of laser pulses IL2, at the first wavelength λ₁. These laser pulses IL2 have an energy that is amplified with respect to the laser pulses IL1.

The amplifying optical fiber 229 of the power amplifier 220 is doped with the same dopant as the doped optical fiber of the injecting laser oscillator 211 so that the transfer function representing the gain of the amplifier is matched to the spectrum of the pulse produced in the oscillator and thus the spectral components of this pulse are preserved.

Within the amplifying optical fiber 229, the energy of the pulse IL2 increases exponentially with propagation distance within the amplifying optical fiber 229, but the central wavelength of the pulse IL2 does not vary.

In at least one embodiment, the length of the amplifying optical fiber 229 is chosen so as to be larger than the distance from which the amplifying optical fiber 229 operates in non-linear regime. Apart from the amplifying effect, the amplifying optical fiber 229 has, on the first train of laser pulses IL1, non-linear effects, i.e. effects that induce non-linear modifications (deformations, introduction of asymmetries, etc.) to the frequency spectrum of the pulses IL1. These non-linear effects are detectable by comparing the frequency spectrum of the pulses IL2 with the frequency spectrum of the pulses IL1 input into the amplifying optical fiber 229. These non-linear effects for example include self-phase modulation and stimulated Raman scattering.

Thus, when the peak power (i.e. the ratio between the energy E and full width at half maximum T_{FWHM} of the pulse) increases and reaches a threshold, the spectrum of the pulse is broadened via a non-linear self-phase modulation effect. Therefore, the pulse is temporally compressed, i.e. its duration is decreased. The peak power then grows increasingly rapidly, this amplifying the self-phase modulation effect. This allows an amplified pulse IL2 of high energy (>100 nJ) and of small duration with respect to an input pulse IL1 to be obtained.

In at least one embodiment, the amplifying optical fiber 229 is thus used in a non-linear regime in which the self-phase modulation effect in an abnormal dispersion regime (β₂<0, β₂ being the dispersion in group velocity expressed in ps²/m) leads to the obtainment of pulses IL2 that are temporally compressed (for example, to a duration equal to or shorter than half the duration of the pulse IL1) with respect to the pulses IL1 output from the injecting laser oscillator 211. This temporal compression is obtained in the amplifying optical fiber 229 provided that the soliton compression point at which the pulse fissions has not been reached.

On account of the amplification, the energy of the pulses amplified in the amplifying optical fiber 229 corresponds to a soliton of order N determined by the following formula:

N²=2πE₂T₀n₂/(|β₂|λ₁A_{eff}) Eq. 1

where E₂ is the energy of the amplified pulse IL2, T₀ the duration of the pulse defined by T₀=T_{FWHM}/(1+2ln(1+2^{1/2})), n₂ the non-linear Kerr index and A_{eff} the effective modal area of the optical fiber. High-order solitons are unstable and fission into N first-order solitons of lower energy (given by equation 1 setting N=1) because of higher order perturbing effects including stimulated Raman scattering, self-steepening and high orders of chromatic dispersion.

In at least one embodiment, the length of the amplifying optical fiber 229 is furthermore strictly smaller than the distance of the point of maximum soliton compression so that high-order perturbing effects, which become important when the spectrum of the pulse broadens, do not cause fission of the pulse IL2 in the amplifying fiber 229.

In at least one embodiment, the amplifying optical fiber 229 of the power amplifier 220 has a very large effective modal area A_{eff} that is larger than 200 λ₁² and for example larger than 500 μm² in the case of an erbium-doped fiber. For example, when the amplifying optical fiber 229 is a single-mode fiber, the maximum energy that the amplified laser pulses IL2 are able to attain increases with the effective modal area of the propagation mode of the amplifying optical fiber 229. As the peak power is proportional to energy for a fixed pulse duration, the larger the effective modal area, the higher the peak power of the amplified pulses. In an amplifying fiber of large effective modal area, it is possible to achieve a very high energy before the self-phase modulation effect affects the spectrum of the amplified pulses (for example by broadening, deformation and/or rupture of the symmetry of the frequency spectrum) in a way such that the pulse will be subject to the perturbing effects and to fission. The distance of the soliton compression point thus corresponds to the propagation distance from which these undesirable effects occur. This distance of the soliton compression point thus depends on the effective modal area of the amplifying optical fiber 229 and on the peak power of the pulse input into the amplifying fiber.

The frequency-shifting device 230 receives the second train of laser pulses IL2 and generates a third train of pulses IL3. More precisely, for each pulse of the second train of laser pulses IL2, a fundamental soliton at a second wavelength λ₂ strictly longer than the first wavelength λ₁ is generated by fission of the amplified pulses IL2 then by Raman self-shifting. The energy of the pulses at the wavelength IL2 is given by equation 1 with N=1. The effective modal area of the frequency-shifting fiber 232 is optimized so that the energy of the pulse IL3 is maximal.

The wavelength λ₂ of the pulses IL3 is strictly longer than the first wavelength λ₁ of the second pulses from which they are generated because stimulated Raman scattering, which is the origin of the frequency self-shifting of the solitons in the frequency-shifting fiber 232, has a dissipative effect in terms of light energy. By virtue of the principle of conservation of total energy, the wavelength λ₂ cannot be shorter than the initial wavelength λ₁.

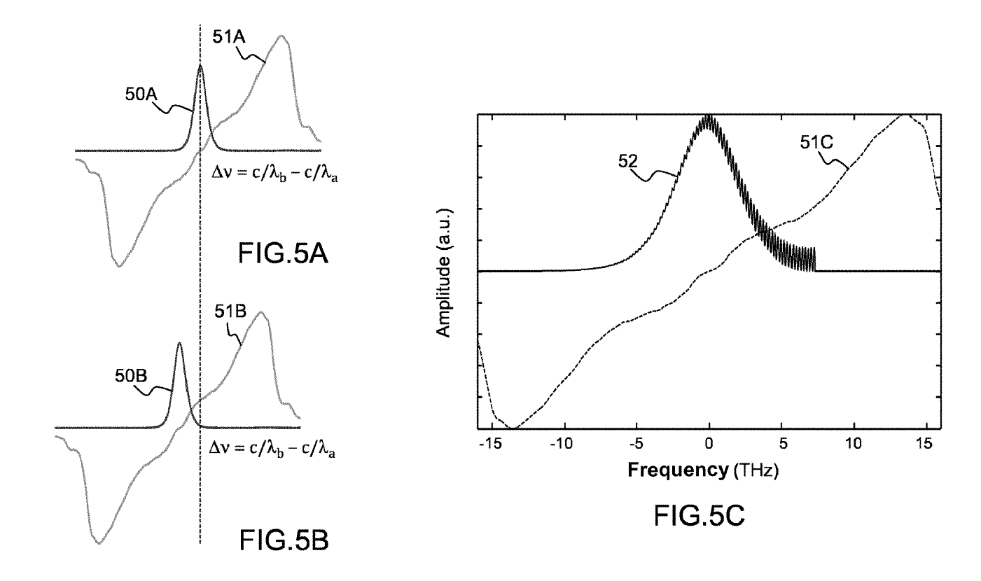

The second wavelength λ₂ is therefore both dependent on the dopant of the amplifying fiber 229, which determines the first wavelength λ₁, and on the Raman scattering within the frequency-shifting fiber 232. Raman scattering adds to the wavelength λ₁ a contribution δλ=λ₁²/c δf where δf is negative and dependent on the Raman susceptibility (see FIGS. 5A and 5B) and where c is the speed of light in free space. Thus, λ₂=λ₁+(λ₁²/c|δf|).

The second wavelength λ₂ is also dependent on the length of the frequency-shifting fiber 232 and on the peak power of the pulse IL2 input into the frequency-shifting fiber 232. The longer the frequency-shifting fiber 232, the larger the frequency shift undergone by the pulse IL2 in this fiber. For a given fiber length, the higher the peak power of the pulse IL2, the larger the frequency shift undergone by the pulse IL2 in this frequency-shifting fiber 232.

In at least one embodiment, the modal parameters of the frequency-shifting fiber 232, including effective modal area and chromatic dispersion, are configured in order to generate at least one fundamental soliton IL3 (N=1) from a pulse IL2 (with N>1) output from the amplifier while maximizing the peak power of the fundamental soliton IL3. In addition, the length of the fiber 232 is chosen in order to tune the central wavelength of the soliton pulse IL3. According to equation (1), the energy E3 of the soliton pulse IL3 generated in the frequency-shifting fiber 232 will be maximal if the modal area and/or the chromatic dispersion of the fiber are maximized. A high chromatic dispersion contributes to spreading of the pulse and, although it then has a high energy, its peak power is not sufficiently increased. The frequency-shifting fiber 232 therefore has a very large modal area, which may be larger than that of the amplifying optical fiber 229, for example larger than 500 μm², so as to receive most of the energy of the second laser pulse IL2.

In at least one embodiment, the amplifying optical fiber 229 is spliced by a splice to the frequency-shifting fiber 232. Any known method for splicing optical fibers may be applied to produce this splice. In particular, electric-arc fusion splicing may be used.

In at least one embodiment, the amplifying fiber 229 and the frequency-shifting fiber 232 are different, but matched to each other in terms of modal area and/or transverse geometry and/or material so as to avoid the loss of power at the splices between these fibers and thus preserve the energy of the pulse input into the frequency-shifting fiber 232. The optimization of a splice between asymmetric fibers is described in more detail for example in the work entitled “Single-mode fiber optics” by Luc Jeunhomme, chapter 3, page 99, Marcel Dekker publishing, New York (1983) ISBN 0-8247-7020-X.

In at least one embodiment, the amplifying fiber 229 and the frequency-shifting fiber 232 are different and asymmetric in terms of modal area, core diameter, core-cladding index difference and/or outside diameter. The energy efficiency of the splice between these two asymmetric optical fibers is in this case optimized by producing an adiabatic taper, i.e. a taper that produces no energy losses. An adiabatic taper is produced by tapering locally the fiber of largest core in order to match the modal areas of the two fibers to be spliced.

In at least one embodiment, the amplifying optical fiber 229 is tapered over all or some of its length in order to significantly increase the diameter of the core (for example in a ratio of 1 to 3 or more between the start and end of the tapered fiber), and thus to significantly increase the energy that the pulse IL2 is able to convey in comparison to a fiber the core diameter of which is constant along its length (translation-invariant).

In at least one embodiment, the frequency-shifting fiber 232 is a tapered fiber the geometric extent of the entrance of which is matched to that of the exit of the amplifying fiber 229.

FIG. 2B illustrates one particular embodiment of a laser source 200B according to the present description.

The laser source 200B shown in FIG. 2B comprises three main components, an injecting laser oscillator 211 based on optical fiber (which fiber is not shown), a power amplifier 220 based on optical fiber 229, and a frequency-shifting device 230 comprising an optical fiber 232. These components are for example those described with reference to FIG. 2A.

The optical fiber 212 output from the injecting laser oscillator 211 conveys the laser pulses IL1 to the power amplifier 220.

In the embodiment illustrated in FIG. 2B, the power amplifier 220 comprises an entrance optical fiber 227, pumping lasers 222, a multimode pump combiner 223, an optical fiber 228 at the exit of the multimode pump combiner 223, and an amplifying optical fiber 229.

The entrance optical fiber 227 receives via the optical fiber 212 the laser pulses IL1 generated by the injecting laser oscillator 211.

The pumping lasers 222 are chosen depending on the wavelength of the spatial multimode infrared radiation that these pumping lasers 222 produce, this wavelength being suitable for producing a population inversion in the rare-earth ions and thus, according to the principle of stimulated emission, for allowing the amplification of the laser pulses IL1. For example, in the case where the rare-earth ion is erbium, the wavelength of the radiation output from the pumping laser may be 979 +/−3 nm or 1532 +/−3 nm.

The multimode pump combiner 223 is suitable for combining in the double-clad single-mode fiber 228 the radiation output from the pumping lasers 222 and the laser pulses IL1 output from the exit fiber 212 of the injecting laser oscillator 211. A continuous background is thus added to the pulses IL1. These pre-amplified pulses IL1 are transmitted via the optical fiber 228 to the amplifying fiber 229. In an identical way to the one described with reference to FIG. 2A with respect to the generation of the pulses IL2 from the pulses IL1, the amplifying fiber 229 generates amplified pulses IL2 from the laser pulses IL1.

In the embodiment illustrated in FIG. 2B, the frequency-shifting device 230 of FIG. 2A comprises a fibered portion 235 and a free-space portion 236. The fibered portion 235 comprises a frequency-shifting fiber 232. The fibered portion may furthermore comprise a cladding light stripper 231 and a collimator 233. The free-space portion 236 for example comprises a filter 234.

The cladding light stripper 231 is configured to remove any residual parasitic radiation output from the pumping lasers 222 and not absorbed in the amplifying fiber 229. The filter 234 is a high-pass filter configured to remove, from the optical wave produced by the frequency-shifting fiber 232, residues of the pulse IL2 at the first wavelength λ₁. The collimator 233 serves to collimate the output optical wave onto the filter 234.

The exit optical component 240 comprises one or more mirrors 241, 242 for conveying through free space the optical pulses output from the filter 234 to the microscope 250.

The optical fiber 228 is for example spliced by a splice 224 to the amplifying optical fiber 229. The amplifying optical fiber 229 is also spliced by a splice 225 to the frequency-shifting fiber 232. Any known method for splicing optical fibers may be applied to produce this splice. In particular, electric-arc fusion splicing may be used.

In at least one embodiment of the laser source 200A or 200B, the injecting laser oscillator 211 is an oscillator that comprises, in addition to the doped internal amplifying fiber described with reference to FIG. 2A, fibers with chromatic dispersion management and that generate frequency-chirped dispersion-managed solitons (DMS). The laser pulses generated by such an oscillator based on fibers with chromatic dispersion management are greatly stretched temporally because of the high group-velocity dispersion of the injecting laser oscillator 211 and have a quasi-Gaussian temporal profile. These pulses furthermore have a broad and quasi-Gaussian spectral profile. These pulses are thus not at the Fourier limit (T_{FWHM}×Δν>0.44 for quasi-Gaussian pulses) and thus have a high frequency dispersion that will possibly be compensated (i.e. decreased or even cancelled out) during the non-linear compression in the amplifying fiber 229. The pulses output from the oscillator with chromatic dispersion management have a sub-300 fs theoretical duration limit, at the wavelength λ₁, so as to be able to be compressed to a sub-picosecond duration, then to give rise to a high-energy pulse IL3. As the energy of a broad-spectrum frequency-chirped pulse may be amplified to higher levels than those obtained for a pulse that is not frequency chirped, it is possible, after amplification and compression in the amplifying fiber 229, to obtain a high peak power, for example by increasing the effective modal area of the frequency-shifting fiber 232. This will make it possible to obtain an effective frequency shift to another wavelength by Raman self-shifting with a frequency-shifting fiber 232 of small length and a higher energy per pulse. In the case of an injecting laser oscillator 211 based on fibers with chromatic dispersion management, the second wavelength λ₂ also depends on the lengths of the dispersion-management fibers of the injecting laser oscillator 211.

In the case of an oscillator based on fibers with chromatic dispersion management, the length of the amplifying fiber 229 may be chosen so that there is therein a temporal compression of the pulses by compensation of the frequency dispersion of the pulses IL1 that is due, on the one hand, to the abnormal chromatic dispersion of the amplifying fiber 229 and, on the other hand, to the self-phase modulation due to the Kerr effect.

In at least one embodiment of the laser source 200A or 200B, the oscillator generates solitons of hyperbolic secant temporal and spectral shape. These solitons are at the Fourier limit (i.e. transform-limited) and characterized by T_{FWHM}×Δν=0.31, i.e. they are the briefest pulses that it is possible to generate with a given spectral intensity profile and they cannot be compressed to a shorter duration than their initial duration.

In at least one embodiment of the laser source 200A or 200B, the injecting laser oscillator 211 is an oscillator that generates pulses that are not frequency chirped, and for example is a soliton oscillator that generates solitons. In at least one embodiment, this soliton oscillator is followed by a normal-dispersion optical fiber that temporally stretches the pulses generated by the injecting laser oscillator 211 to a picosecond duration. This assembly produces frequency-chirped pulses similar to those output from an oscillator with chromatic dispersion management.

In at least one embodiment of the laser source 200A or 200B, the frequency-shifting fiber 232 is a fiber that is selectively absorbent so as to absorb, in the optical wave produced in the frequency-shifting fiber 232, pulse residues at the first wavelength λ₁. In the case of the laser source 200B, the high-pass filter 234 and the collimator 233 may be removed.

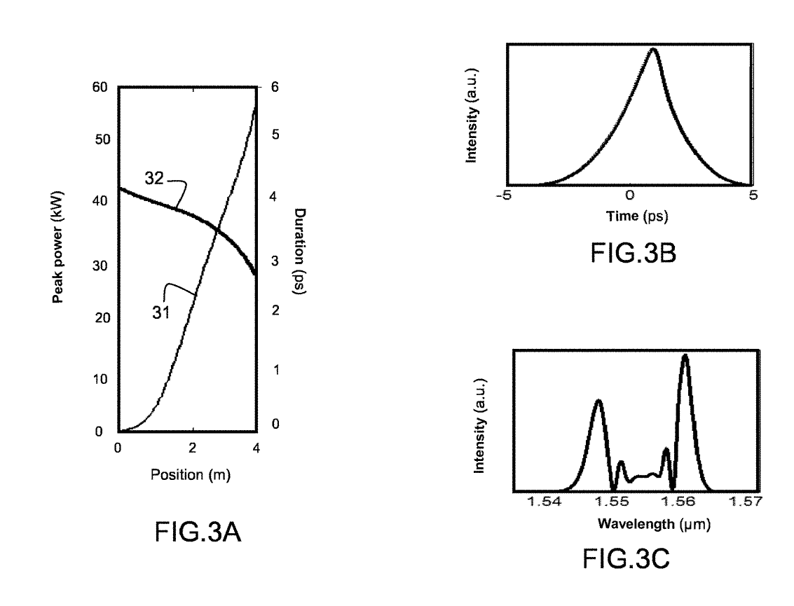

The way in which the length of the amplifying fiber 229 is chosen is illustrated by FIG. 3A, which shows the duration (curve 32) of the laser pulse IL1 and the peak power (curve 31) of the laser pulse IL1 in the amplifying optical fiber 229 of the power amplifier 220 as a function of propagation distance with respect to the point of entry into this amplifying optical fiber 229. In this example, the decrease in the duration of the laser pulse that occurs as the energy of the pulse increases may be seen. The duration of the laser pulse decreases abruptly from about 3 m from the point of entry into the amplifying optical fiber 229, this being the sign that the self-phase modulation has become predominant in the temporal compression process. The energy accumulated by the laser pulse IL2 on exiting the fiber 229 reaches several tens of nJ (about 157 nJ, i.e. a peak power of 57 kW in the example of FIG. 3A) for a full width at half maximum of 3 ps.

FIG. 3B shows the temporal variation in the intensity of the pulse obtained at 3 m from the point of entry into the amplifying optical fiber 229 and FIG. 3C shows the spectrum of the same pulse. Although the pulse is still integral as shown in FIG. 3B, its spectrum is deformed under the effect of self-phase modulation as shown in FIG. 3C. Beyond this point, the laser pulse IL1 fissions into a plurality of elementary soliton waves under the aforementioned perturbing effects. FIG. 3C is thus an illustrative example of a deformation limit on the frequency spectrum, beyond which the pulse loses its integrity, or even fissions. An effect of spreading of the spectrum and a loss of symmetry between high and low frequencies may be seen in FIG. 3C since the spectrum of the pulse input into the amplifying optical fiber 229 is quasi-Gaussian.

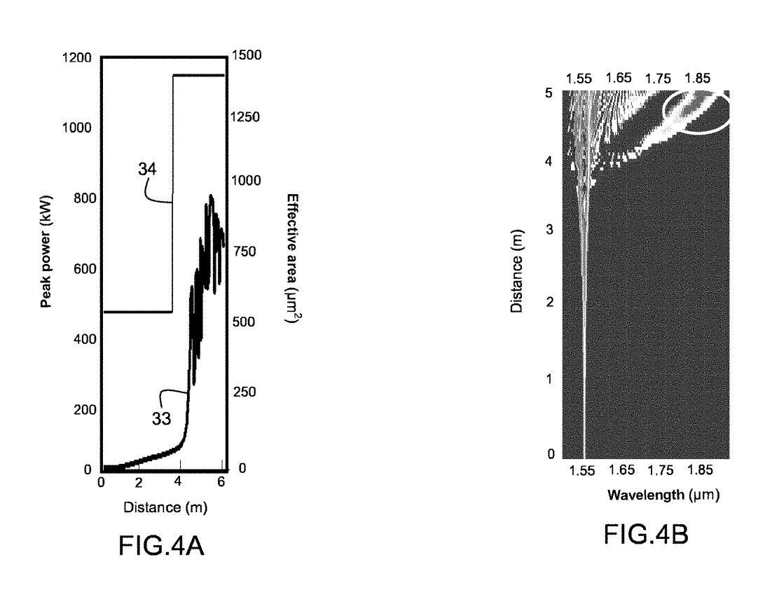

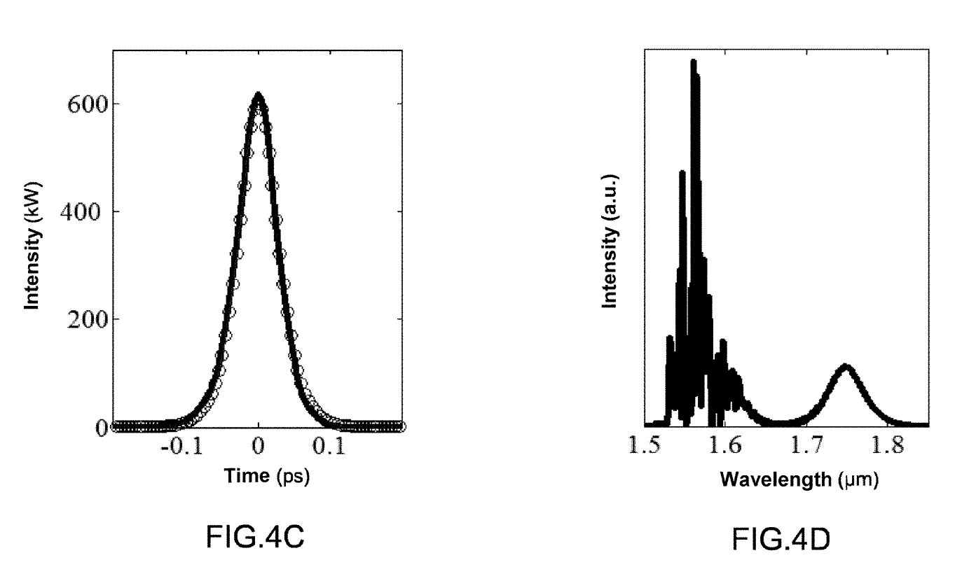

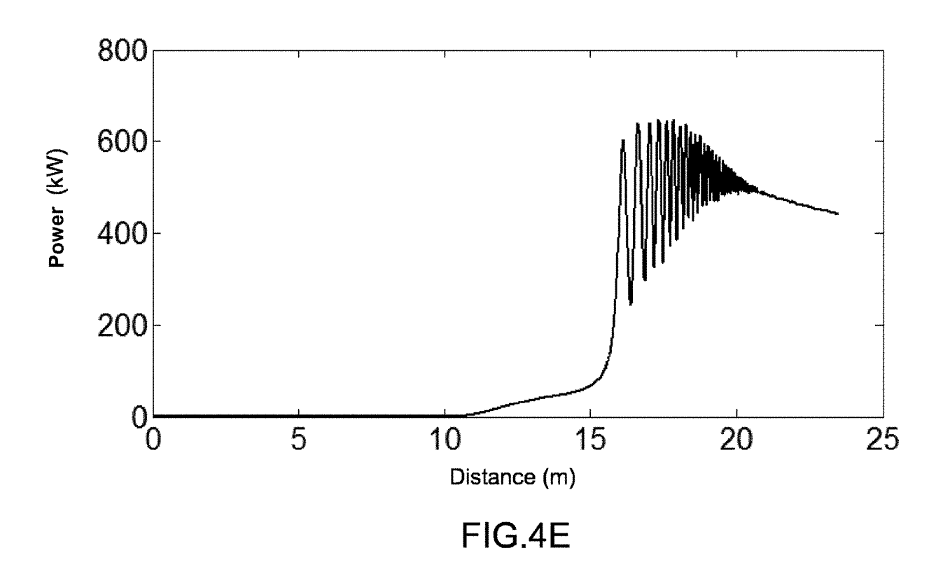

The graph of FIG. 4A illustrates the way in which the effective modal area (curve 34) of the amplifying fiber 229 and the frequency-shifting fiber 232 vary, the jump in the curve 34 corresponding to the passage (at a propagation distance of about 4 m) from the amplifying fiber 229 to the frequency-shifting fiber 232. The graph of FIG. 4A also illustrates the way in which the peak power (curve 33) of the laser pulse IL2 increases in the amplifying fiber 229 then in the frequency-shifting fiber 232 under the effect of multi-soliton compression. It may be seen that, up to a propagation distance of 4 m with respect to the point of entry into the fiber 229, the peak power increases almost linearly (as in FIG. 3A). Between the start of the frequency-shifting fiber 232 and up to approximately a propagation distance of 0.5 m with respect to the point of entry (at a distance of about 4 m) into this frequency-shifting fiber 232, the peak power increases exponentially to a value of 500 kW, as a result of multi-soliton compression. The frequency-shifting fiber 232 is designed so as to obtain a value for the order N of the soliton of close to unity and thus to minimize the number of solitons after fission of the pulse IL2. The order N here has a value of 2.6 (E₂=157 nJ, T_{FWHM}=55 fs, A_{eff}=1250 μm², β₂=2.9×10⁻²⁶ s²/m) for example achieved by maximizing effective modal area. Next, after 0.5 m with respect to the point of entry into the frequency-shifting fiber 232, the pulse IL2 fissions into two fundamental solitons including the soliton pulse IL3, which corresponds to the circled portion at the top right of FIG. 4B.

FIG. 4C shows the temporal variation in the intensity of the pulse IL3 output from the frequency-shifting fiber 232. It may be seen that the pulse has an ultra-brief full width at half maximum, narrower than 100 fs. The peak power of the soliton pulse IL3 output from the frequently-shifting fiber 232 may be determined and is here equal to 600 kW for a full width at half maximum of 100 fs. The circles on the curve of FIG. 4C correspond to the points of a fitted curve (in the present case, a squared hyperbolic secant) showing that the pulse indeed corresponds to a soliton. The curve of FIG. 4D shows the output spectrum of the frequency-shifting fiber 232. The central wavelength of the soliton pulse IL3 is 1.75 μm.

The Raman self-shifting effect is illustrated by FIGS. 5A-5C. FIG. 5A shows the frequency spectrum 50A of a laser pulse before self-shifting in correlation with the curve 51A of the imaginary part of the Raman susceptibility, and FIG. 5B shows the frequency spectrum 50B of a laser pulse after self-shifting, in correlation with the curve 51B of the imaginary part of the Raman susceptibility. An ultra-brief pulse propagating in a physical medium is subject to stimulated Raman scattering. As known, the Raman effect causes a continuous drift in the central frequency of the pulse via an exchange of energy with the phonons of the medium that forms the core of the optical fiber. Via this inelastic light-matter interaction, a photon of wavelength λₐ, i.e. of energy Eₐ=hc/λₐ, where h is the quantum of action or Planck's constant (h=6.62×10⁻³⁴ J·s) and c is the speed of light in free space, is absorbed by the physical medium. A second photon at a lower energy E_{b}<Eₐ is emitted by the Raman medium at a longer wavelength λ_{b}>λₐ. The energy difference, or quantum defect, is transmitted to the physical medium in the form of a particle corresponding to an acoustic vibration in the physical medium, or phonon. The imaginary part of the Raman susceptibility is shown in FIG. 5A, in the case for example of silica as a function of the frequency difference between the two photons at play Δν=c/λ_{b}−c/λₐ). During the interaction via the Raman effect between the physical medium and an ultra-brief pulse of broad frequency spectrum, such as the pulse shown in FIG. 5A, the portion of the spectrum of the pulse for Δν>0 is absorbed whereas the portion of the spectrum of the pulse at Δν<0 is amplified. This corresponds to slippage of the spectrum of the pulse toward low frequencies, as shown in FIG. 5B, i.e. a shift from the center of the pulse to longer wavelengths.

FIG. 5C more precisely shows the frequency spectrum (curve 52) of an ultra-brief pulse propagating in a Raman medium in correlation with the curve 51C of the imaginary part of the Raman susceptibility. The origin of the frequencies (ν=0) is set by convention equal to the frequency of the apex of the frequency spectrum of the pulse. Under this convention, the imaginary part of the spectrum of the Raman susceptibility is centered on the spectrum of the pulse. The positive portion of the spectrum of the Raman susceptibility corresponds to absorption by the material whereas the negative portion of the spectrum of the Raman susceptibility corresponds to an emission. It may be seen that the two spectra superpose and that the portion of the pulse at positive frequencies may be absorbed by the Raman medium. In this example, the absorption maximum is located at 13.2 THz (curve 51C) and the full width at half maximum of the spectrum (curve 52) of this pulse is about 6 THz. Although the maximum of the frequency spectrum is not superposed with the absorption maximum and despite the fact that the width of the frequency spectrum is relatively small, Raman scattering nevertheless produces a frequency shift. Photons at these frequencies will therefore give rise to the emission of photons at frequencies that are negative with respect to the center of the pulse. This transfer of energy from the positive frequencies of the pulse to the negative frequencies of the pulse corresponds to frequencywise slippage of the center of the pulse, i.e. a shift toward longer wavelengths of the apex of the frequency spectrum of the pulse.

During this process of Raman self-scattering, the quantum defect contributes to a decrease in the peak power of the pulse, as observed in FIG. 4E. The peak power decreases gradually with the increase in the distance to the point of entry into the frequency-shifting fiber 232 and as the central wavelength of the pulse is shifted toward longer wavelengths because of the quantum defect related to the interaction, via the Raman effect, between the light and the material.

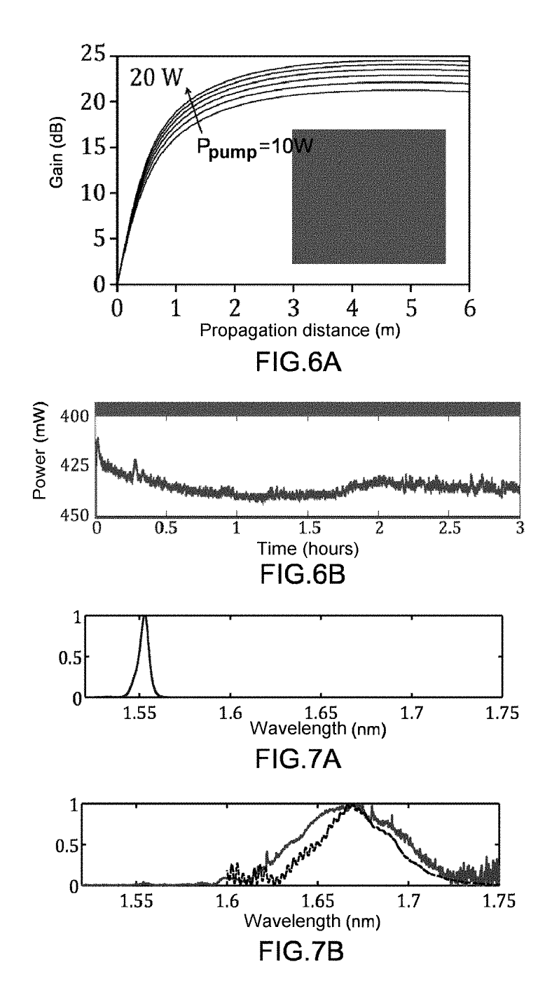

FIG. 6A illustrates the way in which the power gain increases in the amplifying fiber 229 as a function of the distance with respect to the point of entry into this fiber. It may for example be seen that, to obtain a gain of 20 dB, a fiber length of at least 2 m is required. Therebeyond, the increase in the gain becomes increasingly less great: it follows an asymptotic curve with a gain maximum of about 25 dB.

FIG. 6B illustrates the drift in the average power of the train of pulses IL3 measured at the exit of the frequency-shifting fiber 232. It may be seen that the drift in power is very low, about 10 to 15 mW for an average power of 425 mW, i.e. a drift of about 2 to 3% over a period of use of 3 hours. This very low drift is perfectly compatible with the aforementioned applications in the biomedical field.

FIG. 7A is an example of a spectrum of the pulse IL1, as a function of the wavelength λ₁: this spectrum is centered on the wavelength λ₁=1555 nm. FIG. 7B is an example of a spectrum of the pulse IL2 as a function of the wavelength λ₂: this spectrum is centered on the wavelength λ₂=1675 nm, illustrating the wavelength shift. The temporal compression undergone by this pulse may also be seen by comparing FIG. 7A with FIG. 7B, this compression corresponding, in this figure, to a broadening of the spectrum.

The present description also relates to an imaging system comprising a laser system according to any one of the embodiments described in this document, and to a microscope for forming and acquiring an image, using a multi-photon microscopy technique, from ultra-brief pulses produced by means of a laser source according to the present description. The pulses are sent to a zone to be imaged. This zone to be imaged is for example a biological tissue. The pulses cause at a given depth in the biological tissue an excitation of the molecules. An optical beam emitted in response by the zone to be imaged is detected within the microscope. An image may be acquired from the detected optical beam.

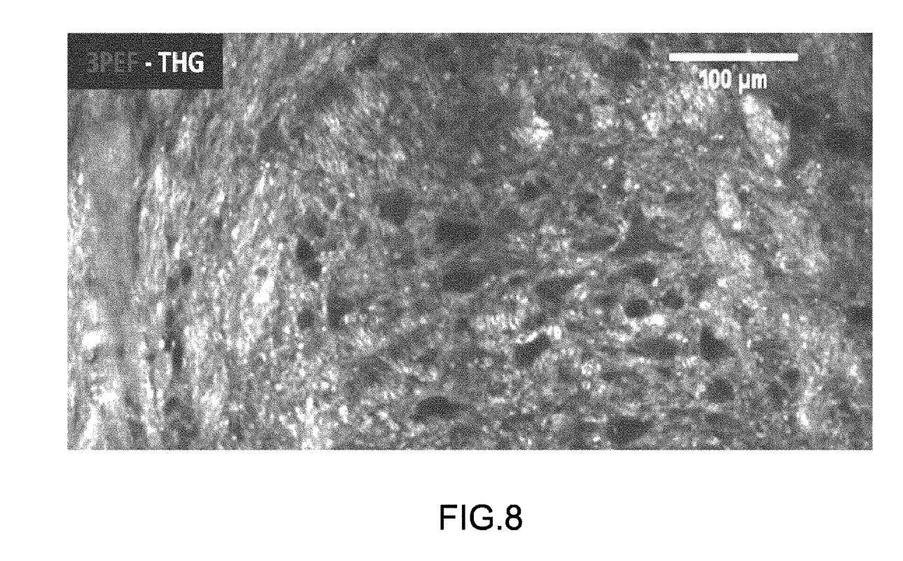

FIG. 8 is an example of an image obtained with an imaging system according to the present description. The figure illustrates an example of three-photon multi-contrast imaging based on three-photon excited fluorescence (3PEF) (red) and third harmonic generation (THG) (white) achieved with the developed laser source on a sample of Rainbow-3 transgenic mouse brainstem tissue (this type of mouse is for example described in the document entitled “Developmental bias in cleavage-stage mouse blastomeres”, by I. Tabansky et al, Current Biology, 2013). The third harmonic imaging allowed a structural image to be obtained without needing to stain this zone of the brainstem. The 3PEF fluorescence signal originated from a marker with which the tissue was stained and which had a higher expression in blood vessels.

Other biological imaging applications may be envisioned. For example an entirely fiber-based laser source according to the present description emitting at a wavelength comprised between 900 and 950 nm may be used for two-photon microscopy when a green fluorescent protein is used as marker. This source will possibly be constructed with neodymium-doped amplifying optical fibers.

Other applications of an entirely fiber-based laser source according to the present description are envisionable when thulium or holmium are used as the rare earth. The radiation IL1 will be obtained at a wavelength of 1.9 to 2.1 μm whereas the radiation at IL2 will have a wavelength longer than 2.1 μm. The ultra-brief pulses of high peak power obtained according to the invention will possibly be used to generate ultra-brief secondary radiation in the UV spectral domain via generation of high-order harmonics.

An entirely fiber-based laser source according to the invention may also be used to generate, via frequency doubling in a suitable non-linear crystal, femtosecond pulses at a wavelength of half of that output from the laser according to the present description. For example if the dopant is holmium, the wavelength of the pulses IL1 and IL2 will be close to 2150 nm whereas the wavelength of the pulses IL3 output from the frequency-shifting fiber will possibly be comprised between 2200 and 2600 nm. By frequency doubling in a suitable non-linear crystal, the wavelength of the pulses will possibly be divided by two and reach the range between 1100 and 1300 nm, which is not currently covered by femtosecond high-peak-power fiber lasers. The high-peak-power pulses thus generated will possibly be used in biological imaging applications implementing THG and 3PEF.

1. A femtosecond laser source comprising:
an injecting laser oscillator based on optical fiber doped with a given dopant, suitable for delivering, via an exit optical fiber, a first picosecond pulse, at a first wavelength Xi;
a power amplifier based on amplifying optical fiber for producing, from the first pulse, a second pulse, at the first wavelength, with an energy that is amplified with respect to the first pulse, the amplifying optical fiber being doped with the same dopant as the optical fiber of the injecting oscillator, and having a length smaller than or equal to the distance of the soliton compression point and larger than the distance from which the amplifying optical fiber operates in non-linear regime; and
a frequency-shifting fiber suitable for receiving the second pulse and generating by Raman self-shifting a fundamental soliton at a second wavelength λ₂ strictly longer than the first wavelength λ₁.

2. The femtosecond laser source as claimed in claim 1, wherein the amplifying optical fiber has a modal area larger than or equal to 200 λ₁².

3. The femtosecond laser source as claimed in claim 1, wherein the frequency-shifting fiber has a modal area larger than that of the amplifying fiber.

4. The femtosecond laser source as claimed in claim 1, wherein the injecting laser oscillator is a fiber oscillator with chromatic dispersion management and that is configured to generate frequency-chirped pulses.

5. The femtosecond laser source as claimed in claim 1, wherein the injecting laser oscillator comprises a soliton oscillator that delivers pulses that are not frequency chirped.

6. The femtosecond laser source as claimed in claim 4, wherein the soliton oscillator is followed by a normal-dispersion optical fiber configured to temporally stretch the pulses generated by the injecting laser oscillator.

7. The femtosecond laser source as claimed in claim 1, wherein the dopant is chosen from the group comprising ytterbium, praseodymium, erbium, thulium, holmium and bismuth.

8. The femtosecond laser source as claimed in claim 1, wherein the amplifying optical fiber and the frequency-shifting fiber are spliced to each other.

9. The femtosecond laser source as claimed in claim 1, furthermore comprising a high-pass filter for removing pulse residues at the first wavelength λ₁.

10. An imaging system comprising a femtosecond laser source as claimed in claim 1, said source being suitable for emitting pulses toward an object located at depth in a biological medium, and a microscope for forming and acquiring an image of the object from fluorescence light backscattered by the object.

11. A method for generating femtosecond laser pulses, comprising
producing, with an injecting oscillator based on optical fiber doped with a given dopant, a first picosecond pulse, at a first wavelength λ₁;
producing, with a power amplifier based on amplifying optical fiber, from the first pulse, a second pulse, at the first wavelength, with an energy that is amplified with respect to the first pulse, the amplifying optical fiber being doped with the same dopant as the optical fiber of the injecting oscillator, and having a length smaller than or equal to the distance of the soliton compression point and larger than the distance from which the amplifying optical fiber operates in non-linear regime; and
producing, by Raman self-shifting, by means of a frequency-shifting fiber suitable for receiving the second pulse, a fundamental soliton at a second wavelength λ₂ strictly longer than the first wavelength λ₁.
